(19) United States
(12) Patent Application Publication
(10) Pub. No.: US 2017/0116879 A1
(43) Pub. Date: Apr. 27, 2017

(54) DIET ADHERENCE SYSTEM

(71) Applicant: Access Business Group International LLC, Ada, MI (US)

(72) Inventors: David W. Baarman, Fennville, MI (US); Matthew K. Runyon, East Grand Rapids, MI (US); Cody D. Dean, Grand Rapids, MI (US)

(21) Appl. No.: 15/401,244

(22) Filed: Jan. 9, 2017

Related U.S. Application Data
(63) Continuation of application No. 14/504,530, filed on Oct. 2, 2014.
(60) Provisional application No. 61/885,773, filed on Oct. 2, 2013.

(51) Int. Cl. G09B 19/00; A61B 5/024; G06F 19/00; A61B 5/00; G01G 19/50; G09B 5/02; A61B 5/053

(52) U.S. Cl. CPC G09B 19/0092; G09B 5/02; A61B 5/024; A61B 5/0537; A61B 5/486; A61B 5/0022; A61B 5/681; G01G 19/50; G06F 19/3475

(57) ABSTRACT

A system and method for providing dietary guidance is provided. The method includes receiving a selection of a health program for an individual, the health program including a dietary regimen, measuring the individual's caloric expenditure and change in body composition or body mass during the individual's participation in the health program, determining adherence to the health program based on the measured caloric expenditure or the measured change in body composition or body mass, identifying a modification to the health program, and informing the individual of the modification. The modification can include nutritional supplements, meals or recipes having a nutritional and/or caloric content tailored to assist the individual in meeting his or her health goals. The method can further include predicting an expected change in body composition or body mass based on the health program and based on the individual's gender, age, height, weight, and other factors.

### FIELD OF THE INVENTION

The present invention relates to weight management systems, and more particularly to automated systems for assisting an individual in setting and/or adhering to diet objectives.

### BACKGROUND OF THE INVENTION

Obesity is a one of the largest health risks in the United States. The Centers for Disease Control estimated that ˜67% of the U.S. adult population is overweight. Individuals on a weight loss program may have a difficult time adhering to a prescribed diet. In at least one study, adherence to diet was determined to be the most critical factor in obtaining weight loss goals, and not type of diet—e.g., Atkins, Ornish, Weight Watchers, and Zone Diets. Experience has revealed that it is common for individuals to set weight loss goals and then to get discouraged and maybe even stop dieting if they do not obtain these goals. In these situations, individuals often times do not understand why they are not obtaining their weight loss goals.

In general weight loss can be achieved when caloric intake is less than caloric expenditure. This idea follows the first law of thermodynamics and can be described by the energy balance equation below, where EI is caloric intake (kcal), EE is caloric expenditure (kcal), and ES is stored energy (kcal) in the form of fat mass (FM) and fat free mass (FFM):

EI−EE=ES (1)

Energy expenditure (or caloric expenditure) can be generally broken down into calories expended through physical exercise, calories expended through resting metabolic rate (“RMR”) and calories expended through diet induced thermogenesis (“DIT”).

There are a wide variety of wearable devices aimed at helping users understand their activity levels and energy expenditure. Often times these devices are marketed as tools for helping with weight loss goals. Many of these devices sync with mobile apps that allow people to manually enter the food they are consuming with the goal of tracking caloric consumption. Over the course of a week an individual may forget, neglect, or just not want to enter some of the food or beverages they have consumed into these manual entry food logs. This inconsistency in data entry, often times, leads to an underreporting of caloric consumption of a period of time.

If an individual is accurately tracking EE with a wearable device and underreporting EI, they may think they should be losing weight (ES), but in reality they are maintaining their current weight or even gaining weight. This phenomena can lead to user frustration and often times cause them to stop dieting and attempting to reach a targeted weigh loss goal.

Weight management is not limited to simply managing weight. In many situations, it is desirable to control body mass index (“BMI”) or the ratio of Fat Mass (“FM”) to Fat-Free Mass (“FFM”), which can be represented by the formula FM/FFM. There are a variety of existing methods for establishing diet and exercise regimens that address body composition or a combination of weight loss and body composition.

It is known to provide a health and information network that is configured to assist a user in improving the user's health and well-being. These types of networks may include a variety of devices that are capable of measuring or otherwise obtaining information that may be relevant to the user's health or well-being, as well as databases for storing information and processors capable of analyzing the information and providing recommendation for improving health and well-being. Network devices may include essentially any device capable of measuring characteristics relevant to health and well-being, such as electronic scales, body composition sensors, blood pressure cuffs, heart rate monitors, sweat sensors, exercise equipment and sleep sensors. Example health and information networks are described in WO/2013/086363, entitled Behavior Tracking and Modification System, filed on Dec. 7, 2012, to David W. Baarman et al., and WO/2014/099255, entitled Systems and Methods for Determining Caloric Intake Using a Personal Correlation Factor, filed Nov. 22, 2013, to Baarman et al, the disclosures of which are hereby incorporated by reference in its entirety.

### SUMMARY OF THE INVENTION

The present invention provides an automated system that assists a user in diet adherence, optionally without manual entry of foods consumed. The system may be configured to assist in meeting weight loss objectives, such as obtaining a defined amount of weight loss or gain over a period of time, and/or body composition objectives, such as changing body composition to achieve a desired body mass index (“BMI”) over a period of time. In one embodiment, the system includes a processor that predicts weight loss at different points in time over the length of a diet (such as daily), a weight measurement device that measures actual weight at those points and a processor that recommends that the user continue to adhere to the diet or modify the diet based on a comparison of actual weight loss with predicted weight loss. For example, if the user has not achieved the expected weight loss or body composition changes at a given period of time, the system may direct the user to modify the user's diet or exercise regimen on a going forward basis to compensate for any shortcomings.

In one embodiment, a method is provided. The method includes a) receiving a selection of a weight loss program for the user, the weight loss program including a dietary regimen, b) measuring the user's caloric expenditure and change in body composition or body mass during the user's participation in the weight loss program, c) determining adherence to the weight loss program based on the measured caloric expenditure and the measured change in body composition or body mass, d) identifying a modification to the dietary regimen, and e) informing the individual of the modification. Modifying the dietary regimen can include recommending one or more nutritional supplements, meals or recipes having a nutritional and/or caloric content tailored to assist the individual in meeting his or her weight loss goals.

In another embodiment, a system is provided. The system includes a first sensor adapted to measure a caloric expenditure, a second sensor adapted to measure body composition or body mass, and a computer adapted to perform the following steps based on the measured caloric expenditure and the measured body composition or body mass: a) determine an expected body composition or body mass, b) compare the measured body composition or body mass with the expected body composition or body mass, and c) recommend a modification of a prescribed dietary regimen based on a departure of the measured body composition or body mass from the expected body composition or body mass. The first device can include a wearable device, the second device can include a weight scale, and the computer can include a cloud server that is remotely located with respect to both of the first device and the second device.

In one embodiment, the system predicts weight loss over the length of the diet at the outset using estimations of energy expended through physical activity, resting metabolic rate (“RMR”) and diet induced thermogenesis (“DIT”) to predict weight loss. In one embodiment, the system includes one or more devices for tracking energy expended by the user during an initial tracking period, for example, one week, to assist in predicting energy expended through physical activity. For example, the system may include a wearable device that includes sensors for tracking energy expended through physical activity during the tracking period. Based on the measured physical activity during the initial tracking period, the system may determine an average daily energy expended through physical activity to be used in making weight loss predictions. In one embodiment, the system may continue to track physical activity during the diet. If the actual energy expended through physical exercise does not sufficiently match the estimated energy expended through physical exercise used in creating the weight loss predictions, the system may revise the weight loss model to account for the difference.

In one embodiment, the system collects or otherwise obtains additional information that may be relevant to energy expenditure and therefore helpful in making accurate weight loss predictions. For example, the user's gender, age, height, weight and ratio of fat mass to fat-free mass may be relevant to RMR. This information may be input into the system by the user. To reduce the risk of error, weight may be obtained and provided by a scale that is capable of communicating directly with the system. Similarly, height may be obtained and provided by a height measuring device that is capable of communicating directly with the system. The system may determine body mass index (“BMI”) through the height and weight measurements using the formula: BMI=Height/Weight². Additionally or alternatively, the system may determine the ratio of fat mass (“FM”) to fat-free mass (“FFM”) using bio-impedance sensors or other devices capable of providing such information. The system may collect or otherwise obtain additional information that may be relevant to making accurate predictions of weight loss or change in body composition that may be useful in setting a healthy and realistic diet objective for the user, such as average resting heart rate of the user, average blood pressure of the user, average amount of daily sleep, average amount of salt in sweat and average hydration level of the user. For example, the system may include a heart rate monitor that may be used to make more accurate measurements of energy expenditure or a hydration sensor that may be used to make more accurate measurements of body composition.

In one embodiment, the system is configured to provide a healthy and realistic diet objective for a user, such as a recommended weight loss objective or a recommended body composition objective. The diet objectives may be selected based on ideal weight and body composition numbers for the user based on prior clinical determinations.

In one embodiment, the system is connected to a larger network of devices that collect and store user information that may be relevant to the health and well-being of the user. In this embodiment, the system may be configured to obtain from one or more devices within the network additional information that may be relevant to formulating healthy and realistic objectives for the user. The network of devices may be connected via the internet or other networking technology. The system may communicate directly or indirectly with devices in the network to transmit and/or receive information from other devices. The network of devices may include a database that contains information relating to the health and well-being of the user, as well as tracking devices that are configured to collect information that may be relevant to the health and well-being of the user. The database may include information specific to the user or general information relating to a collection of individuals. The tracking devices may include essentially any type of device capable of measuring or otherwise obtaining information of potential relevance to health and well-being, such as exercise equipment, nutritional supplement dispensers, sleep monitoring devices, stress monitoring devices and devices configured to collect information concerning food consumption. When used, food consumption information may include essentially any characteristic of consumed food that has the potential to impact health and well-being, such as caloric intake and/or nutritional content. For example, information relating to the amount of fat and/or protein in consumed food may be particularly useful in meeting body composition objectives.

In one embodiment, the system is configured to provide a user with recommendations not specific to diet adherence that may assist in achieving the weight loss or body composition objectives or that may assist in improving overall health and well-being. In such embodiments, the system may monitor average resting heart rate, average blood pressure, average hydration levels or other factors that may be relevant to health and well-being. In these embodiments, the system may analyze all of the available information and make recommendations specific to the user. For example, the system may recommend changes in the types of foods that are consumed, such as recommend a low-sodium diet or a diet that is high in protein. The system may even recommend specific recipes or suggest how to modify existing recipes to implement the recommended dietary changes. As other examples, the system may recommend an exercise regimen, may recommend increased amounts of sleep or may recommend increased water consumption.

The present invention provides a simple and effective system that is capable of assisting a user with diet adherence without requiring the user to input information regarding food consumption. This helps to eliminate errors created by inaccurate or incomplete entry of food consumption information. In those embodiments that provide recommended diet objectives, the system also assists in setting healthy and realistic objectives to avoid the health risk and disappointment that may result from inappropriate objectives. The system may be configured to collect information needed to provide recommendations in an automated manner to facilitate use of the system. In some embodiments, the system may be capable of communicating with a health and wellness network including a plurality of health and wellness devices configured to assist a user in improving health and well-being. In such embodiments, the system may be capable of leveraging resources available within the health and wellness network. Further, the system can be configured to contribute its information and other resources to the network of devices to assist those devices in performing their functions.

These and other objects, advantages, and features of the invention will be more fully understood and appreciated by reference to the description of the current embodiment and the drawings.

Before the embodiments of the invention are explained in detail, it is to be understood that the invention is not limited to the details of operation or to the details of construction and the arrangement of the components set forth in the following description or illustrated in the drawings. The invention may be implemented in various other embodiments and of being practiced or being carried out in alternative ways not expressly disclosed herein. Also, it is to be understood that the phraseology and terminology used herein are for the purpose of description and should not be regarded as limiting. The use of “including” and “comprising” and variations thereof is meant to encompass the items listed thereafter and equivalents thereof as well as additional items and equivalents thereof. Further, enumeration may be used in the description of various embodiments. Unless otherwise expressly stated, the use of enumeration should not be construed as limiting the invention to any specific order or number of components. Nor should the use of enumeration be construed as excluding from the scope of the invention any additional steps or components that might be combined with or into the enumerated steps or components.

### BRIEF DESCRIPTION OF THE DRAWINGS

FIG. 1 is a plot of Actual Body Mass versus Predicted Body Mass, where Predicted Body Mass is determined according to prior art equation (2) herein.

FIG. 2 includes two plots correlating energy expenditure and weight loss over successive two week time intervals.

FIG. 3A is a flow chart depicting a method for recommending dietary modifications as part of a weight loss program.

FIG. 3B is a flow chart depicting a method for recommending health plan modifications as part of a weight loss program.

FIG. 4 is a schematic representation of a system Of the present invention to determine dietary adherence as part of a health plan.

FIG. 5 illustrates a device network for a system adapted to determine dietary adherence as part of a health plan.

FIG. 6 illustrates a first graphical application for a mobile device including information relating to adherence to a user-designated health plan.

FIG. 7 illustrates a second graphical application for a mobile device including information relating to adherence to a user-designated health plan.

FIG. 8 is a diagram of a nutrition management system according to one embodiment of the present invention.

FIG. 9 depicts various devices configured for use in one embodiment of the present invention.

FIG. 10 depicts various devices configured for use in one embodiment of the present invention in conjunction with web-based cloud computing.

FIG. 11 is a graph according to one embodiment in which energy expenditure attributable to physical activity is tracked over a period of time.

FIG. 12 are charts according to one embodiment in which caloric intake and caloric expenditure are shown.

FIG. 13 is a chart according to one embodiment depicting changes in intake of different macronutrients over a twenty-four hour period.

FIG. 14 is a chart showing body mass index ranges that may be considered healthy.

FIG. 15 is a chart and a table showing examples of data collected by one embodiment of the present invention to determine an average daily energy expenditure.

FIG. 16 is a plot of a model weight loss prediction according to one embodiment of the present invention.

FIG. 17 shows charts according to one embodiment of the present invention, and representing different weight outcomes over different adherence scenarios.

FIG. 18 is a chart according to one embodiment in which an individual's weight loss may be analyzed in terms of fat free mass relative to fat mass.

FIG. 19 includes a schematic of microbiome and genetic analyses as an aid to understanding genetic and microbial predispositions.

FIG. 20 includes a one-year microbiome assessment to provide a responsive indicator for the purposes of measuring diet adherence.

### DESCRIPTION OF THE CURRENT EMBODIMENTS

A system and method in accordance with an embodiment of the present invention enables tracking of the user's adherence to a predefined health metric in an automated manner. In one embodiment, the system and method may track characteristics of a user for a period of time to develop a user profile. Characteristics of the user, for example, may include one or more of weight, activity levels, heart rate, blood pressure, average fat mass (FM), free fat mass (FFM), and hydration level. It should be understood that the present invention is not limited to these characteristics, and that any type of user characteristic may be tracked in developing the user profile. Based on the user profile, the system and method may form one or more health metrics or objectives selected to achieve user adherence, and provide one or more recommendations for achieving the objections. The one or more objectives may be selected in part based on the likelihood of user adherence. The one or more health metrics or objectives may also be selected to be healthy or within health parameters specific to the user, such as the age and weight of the user.

As discussed below, the system and method of the present invention can include measurements of one or more values. These values can include for example caloric expenditure, caloric intake, body mass, body composition, body mass index, ratio of fat mass to fat-free mass, heart rate, height, weight, temperature, and a change over time to any of the foregoing. As the term is used herein, to “measure” a value means to directly or indirectly determine at least one of an actual value and an estimated value. For example, to “measure” a caloric expenditure includes directly or indirectly determining an actual caloric expenditure or an estimated caloric expenditure, optionally in conjunction with a method for determining adherence with a weight loss program. As also used herein, a “measured” value includes at least one of the actual value and the estimated value. For example, a measured caloric expenditure includes an actual caloric expenditure or an estimated caloric expenditure as determined either directly or indirectly, optionally in conjunction with a method for determining adherence with a weight loss program.

Within the selected or predefined framework of health metrics, the system and method may track a user's adherence to the health metrics, or more particularly a health plan having one or both of a dietary regimen and an exercise regimen. The system and method may continue to track characteristics of the user to determine automatically whether the user is adhering to the one or more health metrics or objectives. In one embodiment, adherence may be determined without manual entry of foods consumed by the user, potentially avoiding discrepancies caused by user error or deception in the manual entry process. If it is determined there is a low degree of adherence to the one or more health metrics, suggestions may be provided to help the user to realistically achieve the one or more health metrics.

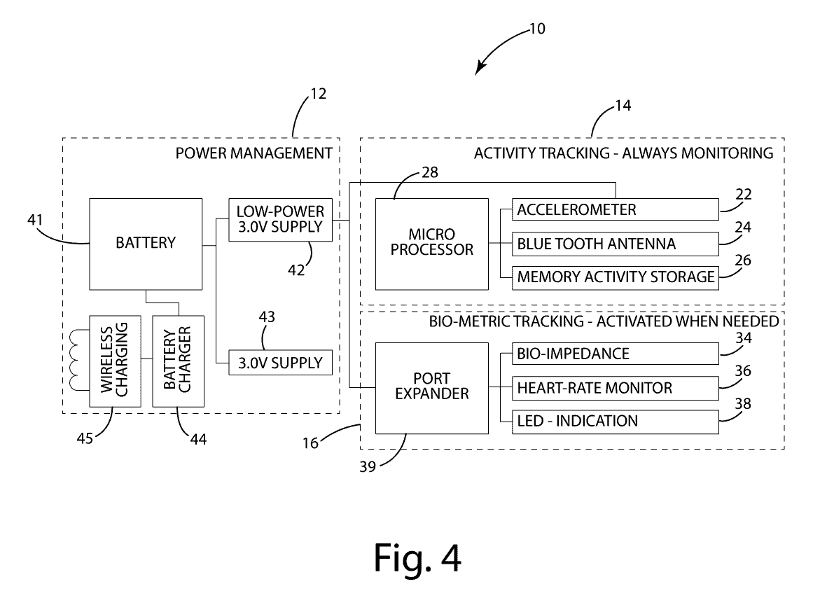

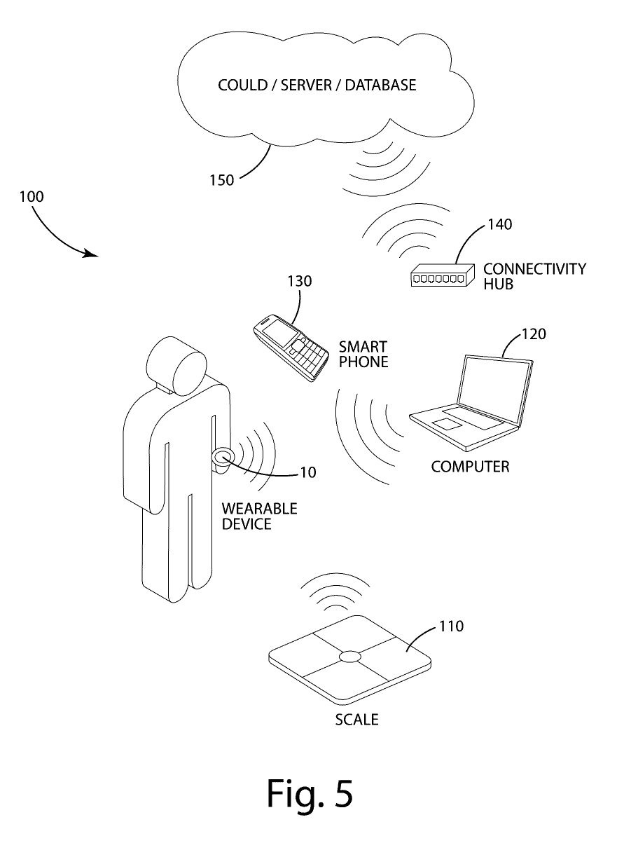

As described herein, the system and method according to an embodiment of the present invention tracks one or more user characteristics. Some of these characteristics may be tracked or associated with a user through use of a personal device, such as the personal device shown in FIGS. 4 and 5, and generally designated 10. The personal device 10 may be carried or worn by the user, and may enable association between the user and other components of the system. The personal device 10 may include one or more of a variety of sensors, data storage, communication circuitry, a user interface, and processing units. As an example, the personal device 10 may be similar to the personal device 10 described in WO2013/086363, entitled Behavior Tracking and Modification System, filed on Dec. 7, 2012, to David W. Baarman et al.—the disclosure of which is hereby incorporated by reference in its entirety.

As also shown in FIG. 5, the personal device 10 may be part of a larger system (or network) of products or components that collect information about user activities, such as diet, exercise and other factors that may be relevant to health and well-being. By collecting this information, the system may be able to assist a user in making choices that improve health and well-being. It is well known that by tracking consumption of food, water, and nutrition and activity, we can get a better picture of our health needs. The personal device 10 represents one aspect of this system but helps to build one element of a larger view of a personal health plan. The system can include a scale 110, a personal computer 120, a smartphone 130, a connectivity hub 140, and/or a remote server 150. The personal device 10 may be configured to communicate with the Internet or other system components 110, 120, 130, 140, 150 using wireless communications, such as WiFi or low energy Bluetooth. The communications capability may allow the personal device 10 to transmit and/or receive personal health information for a user. The larger system may include a variety of components, including food, supplements, or beverage dispensers, or a combination thereof. For example, the larger system may include the food supplement dispenser or beverage dispenser described in U.S. Patent Application Publication 2013/0110283, entitled Pill Dispenser, filed Apr. 25, 2012, to Baarman et al.—the disclosure of which is hereby incorporated by reference in its entirety.

In the illustrated embodiment of FIG. 4, the personal device 10 may include power management circuitry 12, activity tracking circuitry 14, and biometric tracking circuitry 16. It should be understood that the personal device 10 in embodiments contemplated herein may include a subset of these components, one or more additional components, or a combination thereof. Further, the personal device 10, or portions thereof, may be integrated into components of a larger system. Although not shown, the personal device 10 may include a user interface that enables a user to provide inputs and control operation of the personal device 10.

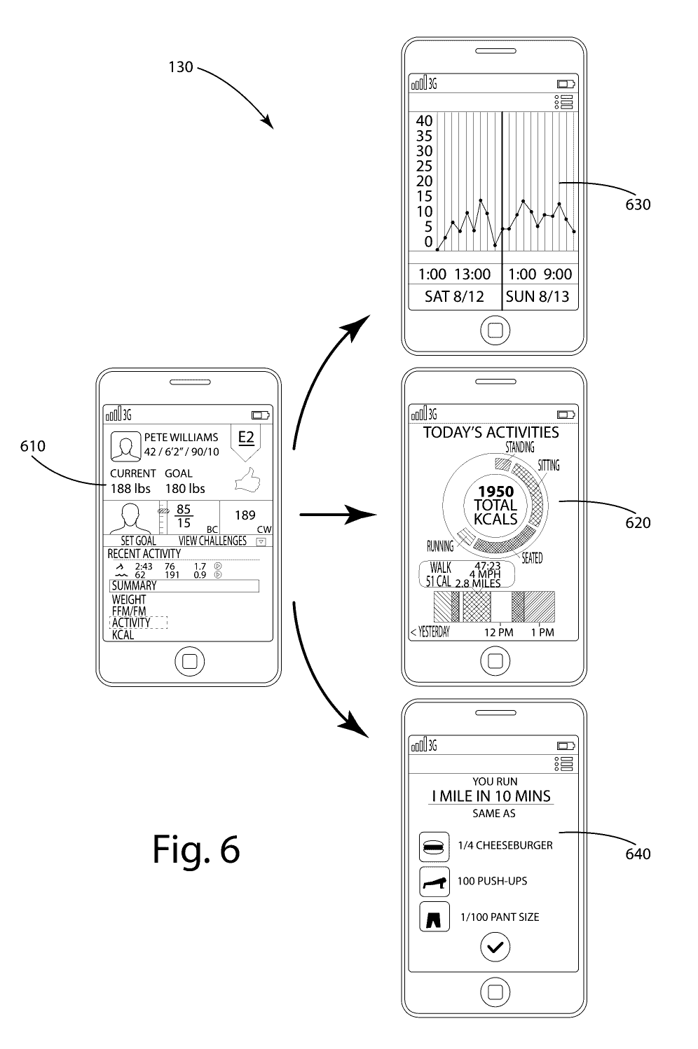

The activity tracking circuitry 14 may include a processor 28, communication circuitry 24, memory 26, and one or more sensors 22. The processor 28 of the activity tracking circuitry 14 may operably couple with the communication circuitry 24, memory 26, and the one or more sensors 22 to track activity of a user associated with the personal device 10. The processor 28 may obtain information from the one or more sensors 22, and use this information as a basis for performing one or more steps according to an embodiment described herein. In one embodiment, the information received from the one or more sensors 22 may be stored in the memory 26. The processor 28 may also interface with the communication circuitry 24 to receive information from external sources, such as information related to the user of the personal device 10 from the devices 110, 120, 130, 140, 150 illustrated in FIG. 6. For example, the communication circuitry 24 may be a Bluetooth interface configured to receive and transmit data and information within the system and to enable user interaction with the system. The information received from external sources may also be used as a basis for performing one or more steps according to an embodiment described herein. Alternatively or additionally, the communication circuitry 24 may enable the personal device 10 to transmit information related to the user, including information obtained from the one or more sensors 22, from the personal device 10 to components in a larger system. Using the information communicated from the personal device 10, the components may perform one or more steps according to an embodiment described herein.

In the illustrated embodiment of FIG. 4, the one or more sensors 22 may include an accelerometer, such as a 3-axis accelerometer. The accelerometer may enable the personal device 10 to monitor movement and activity levels of the associated user. In one embodiment, the one or more sensors 22 may enable continuous monitoring of a user's activity levels. It should be understood, however, that the present invention is not limited to continuous monitoring, and that, additionally or alternatively, the one or more sensors 22 may be configured to monitor a user's activity level intermittently, periodically, or event-based, or a combination thereof, as desired depending on the application.

The activity tracking circuitry 14 may also interface with one or more biometric sensors of the biometric tracking circuitry 16. For example, the processor 28 may be operably coupled to expansion circuitry 32 of the biometric tracking circuitry 16 that allows the processor 28 to interface with one or more additional sensors, such as a bio-impedance sensor. In this way, the personal device 10 may obtain or sense biometric information related to the user. The processor 28 may interface with the biometric tracking circuitry 16 to obtain biometric information when desired or event-based such that sensors of the biometric tracking circuitry 16 can potentially avoid a continuous draw of power from the power management circuitry 12. Alternatively or additionally, the biometric tracking circuitry, or components thereof, may be configured for continuous, intermittent, or periodic monitoring of the user. In an alternative embodiment, the one or more sensors described in connection with the biometric tracking circuitry 16 may interface directly with or be operably directly coupled to the activity tracking circuitry 14.

In the illustrated embodiment of FIG. 4, the biometric tracking circuitry 16 may include bio-impedance measurement circuitry 34. The biometric tracking circuitry 16 may include bio-resonance measurement circuitry in addition to our alternative to the bio-impedance measurement circuitry 34. The bio-impedance measurement circuitry 34 or bio-resonance measurement circuitry, or both, may enable the device to sense information related to a body composition of the user. Based on this body composition information, the processor 28, or another component, may make a determination regarding Fat Mass and Fat Free Mass.

The biometric tracking circuitry 16 may include a heart rate monitor 36 capable of providing an output indicative of the user's heart rate. This heart rate information may be analyzed in conjunction with sensor output related to an activity level of the user to, for example determine a resting heart rate. Although described in connection with a heart rate monitor 36 and bio-impedance measurement circuitry 34 in the illustrated embodiment of FIG. 4, the biometric tracking circuitry 16 may be configured differently. For example, the biometric tracking circuitry 16 may include one or more additional biometric sensors, such as a temperature sensor, blood pressure sensor, and a hydration level sensor. And, the biometric tracking circuitry 16 may not include bio-impedance measurement circuitry 34 or the heart rate monitor 36, or both. The biometric tracking circuit 16 may additionally include a port expander 39 electrically coupled between the processor 28 and the sensors 34, 36.

The personal device 10 may include power management circuitry 12 that controls or manages supply of power to components of the personal device 10, such as the activity tracking circuitry 14 and the biometric tracking circuitry 16. The power management circuitry 12 may include a battery 41 and one or more regulators 42, 43. In one embodiment, depending on the operational needs of components of the personal device 10, the power measuring circuitry 12 may include one or more regulators 42, 43 capable of providing different power outputs. For example, the power measuring circuitry 12 may include a low-power 3 V supply 42 capable of providing regulated power from the battery 41 to the processor 28, the one or more sensors 22, communication circuitry 24, memory 26, and the expansion circuitry 32. And, the power measuring circuitry 12 may include another 3 V regulator 43 coupled to the battery 41 and purposed for supplying power to the bio impedance measurement circuitry 34.

The battery 41 of the personal device 10 may be charged in a variety of ways. In the illustrated embodiment, the power management circuitry 12 may include wireless power circuitry 45 and battery charging circuitry 44. The wireless power circuitry 45 may include a secondary or a receiver capable of receiving power wirelessly or without direct electrical contacts. For example, the wireless power circuitry 45 may receive power from a transmitter via an inductive coupling between a primary of the transmitter and the secondary. Alternatively or additionally, the power management circuitry 12 may include a charging interface capable of receiving power from a supply via direct electrical contacts. Power received in the power measuring circuitry 12 may be utilized by the charging circuitry 44 to charge the battery 41.

The personal device 10 in the illustrated embodiment of FIG. 4 may include user feedback circuitry 38 that allows the personal device 10 to provide feedback or information to the user. For example, the user feedback circuitry 38 may include one or more LEDs capable of being selectively activated based on one or more parameters determined by or received in the personal device 10. As another example, the user feedback circuitry 38 may include a visual display that communicates information to the user. In the illustrated embodiment of FIG. 4, the user feedback circuit 38 is included in the biometric tracking circuitry 16, but it should be understood that the user feedback circuitry 38 may be incorporated or interface with other circuitry or components of the personal device 10.

Turning now to the illustrated embodiment of FIG. 5, the personal device 10 may be used in conjunction with a system of components, designated 100, to achieve user tracking or to provide recommendations, or both. The system 100 may include a variety of devices configured for various purposes, including communicating with the personal device 10, providing information to the user, relaying information from one device to another, and sensing information related to the user. For example, the system 100 may include a computer 120 or a remote device 130 (e.g., a smart phone), or both, that is capable of communicating with the personal device 10 to receive and transmit information, and capable of providing information, such as one or more recommendations, to the user and obtaining user feedback. The communication hub 140 may relay information from one or more devices in the system 100 to one or more other devices in the system 100. For example, the communication hub 140 may enable the computer 120 or the remote device 130, or both, to communicate with an external server 150, such as a cloud store or a database, or a combination thereof. As another example, the communication hub 140 may receive information related to the user, such as the user's weight from a scale 110, and pass this information along to the external server 150 for storage. Alternatively or additionally, the communication hub 140 may also relay information to and from the personal device 10.

In the illustrated embodiment of FIG. 5, one of the devices in the system 100 is a scale 110 capable of weighing the user, and communicating the weight of the user to another device of the system 100, such as the personal device 10 or the remote device 130, or both. This weight information may be used in conjunction with a method according to an embodiment described herein to track adherence to one or more objectives, including, for example, predicting a weight or diet metric of the user.

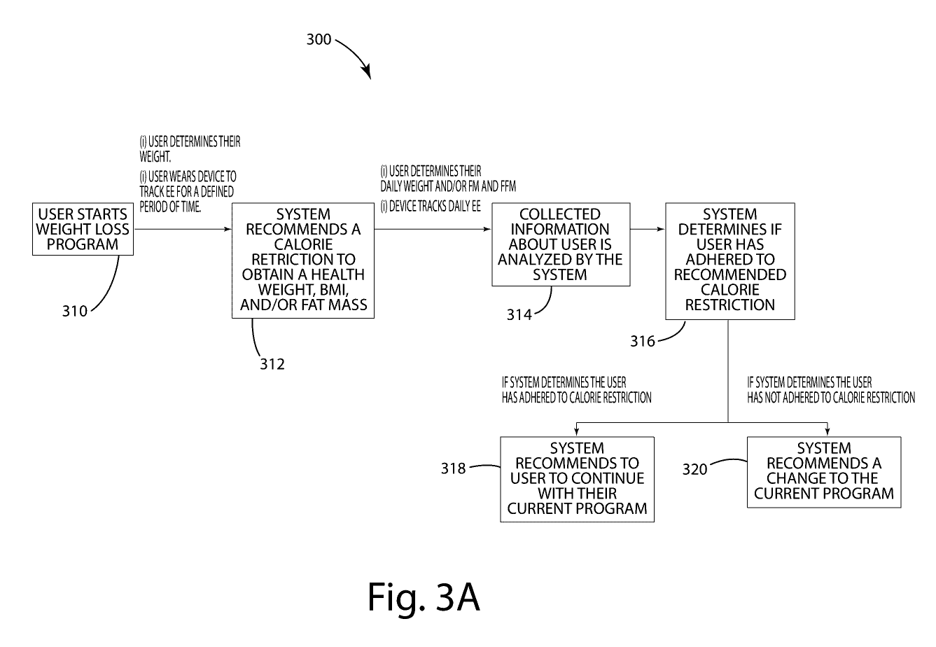

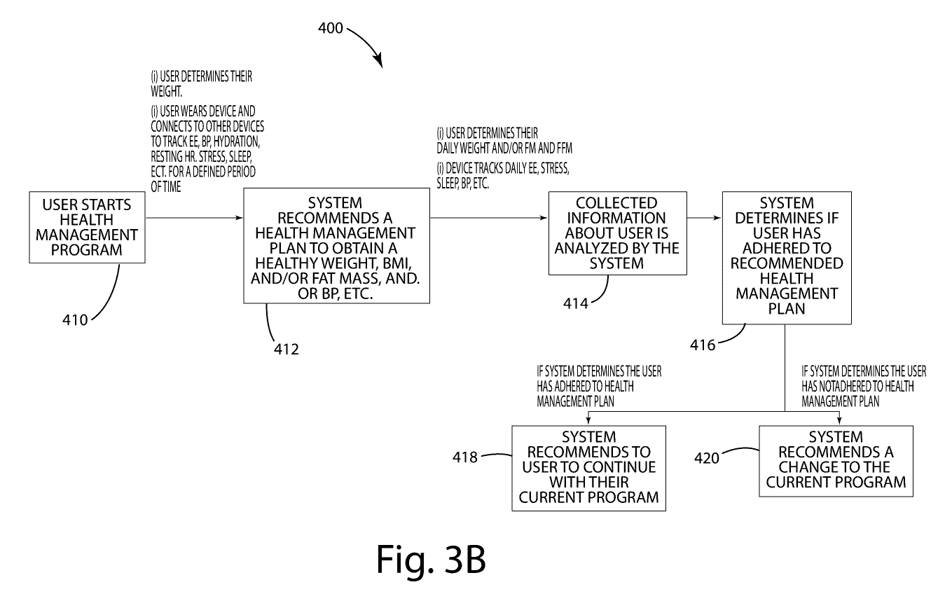

A method of developing a weight loss objective and assisting the user in achieving the weight loss objective will now be described with respect to the illustrated embodiment of FIG. 3A. As shown, the method designated 300 may be implemented in a system to track information related to or characteristics of a user to develop the weight loss objective. The system may continue to track information about the user to determine adherence to the weight loss objective, and provide one or more recommendations to help the user to achieve the weight loss objective.

Starting with step 310, the user may initiate the weight loss program according to the method 300 within the framework of a system 100, including a personal device 10. Although described in connection with the system of FIG. 5, it should be understood that the method 300, or one or more steps thereof, may be implemented in any system or component described herein. For an initial period of time (e.g., a week), the weight of the user is determined on a periodic basis, such as on a daily basis, and the user wears the personal device 10. The weight of the user may be determined using a scale, such as the scale 110, which automatically reports the user's weight to a component within the system 100, such as the personal device 10 or the external server 150. Alternatively, the user may manually enter their weight into the system—though, as mentioned above, there may be potential for user error with manual entry. Thus, automated reporting of the user's weight via the scale 110 may help the system 100 to track the user's weight without this potential for user error.

In addition to monitoring the user's weight during the initial period, the system 100 may also track activity levels related to energy expenditure based on output from the one or more sensors of the personal device 10, such as accelerometer information obtained while the user wears the personal device 10. The system 100 may also track a variety of additional characteristics or obtain additional information related to the user during the initial period, including tracking one or more of body composition (e.g., FM/FFM ratio), BMI (Body Mass Index), age, gender, blood pressure, hydration, resting heart rate, stress, and sleep. The personal device 10, as outlined above, may include one or more sensors capable of tracking this information. Information obtained during the initial period may also include family history, or DNA analysis, indicative of potential medical issues or a predisposition toward medical conditions, such as high blood pressure.

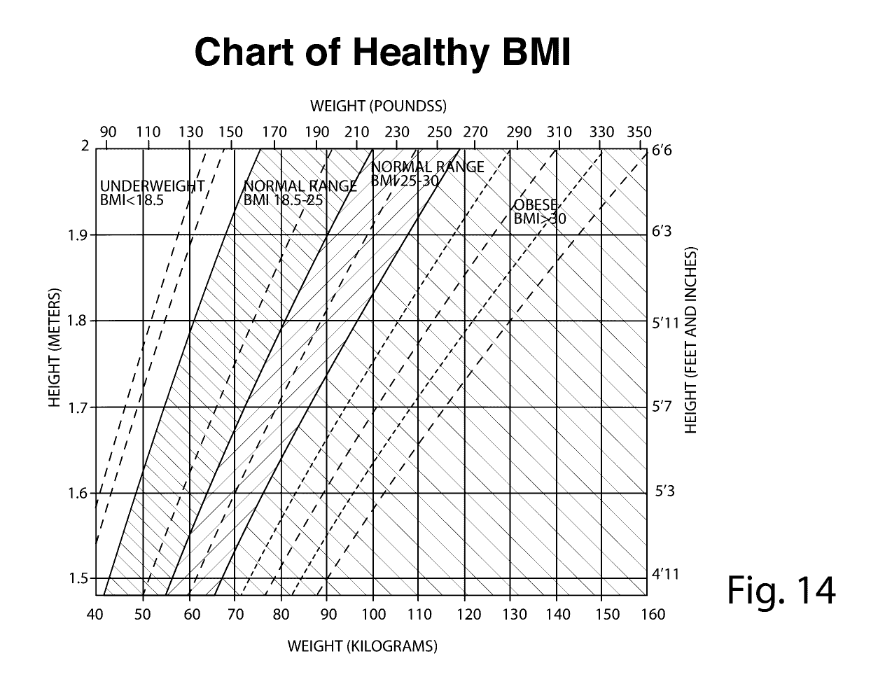

Based on information and data collected about the user, the system 100 may develop one or more objectives to achieve a healthy target weight, including a caloric restriction recommendation (dietary regimen) or an increased activity recommendation (exercise regimen), or both. Step 312. For example, an objective may be a diet objective selected based on ideal weight. Additionally or alternatively, the objectives may be related to achieving one or more of a target BMI and a target Fat Mass or body composition. As an example, the system 100 may recommend a healthy weight loss target, such as losing 20 pounds in 4 months, based on factors or characteristics related to the user, including average daily energy expenditure, age, gender, BMI, and body composition. And, based on the healthy weight loss target, the system 100 may provide a caloric restriction recommendation of 200 fewer daily calories or an increased activity recommendation to exercise 20 minutes per day. The healthy weight loss target, the caloric restriction recommendation, or the increased activity recommendation, or a combination thereof, may be determined by entering user related factors into a table or database of information. In other words, the table or database of information may correlate factors related to the user to a healthy weight loss target, a caloric restriction recommendation, or an increased activity recommendation, or a combination thereof. The table or database of information may also account for the likelihood of user adherence such that, for example, the system 100 may avoid providing unachievable or unhealthy recommendations or recommendations that the user would consider unreasonable. For example, the database may utilize information based on a healthy BMI for a given height and weight, such as those identified in FIG. 14. The recommendations, such as a caloric restriction, may be based on prior clinical determinations. For example, a caloric restriction recommendation may not result in a diet of less than 1200 kcal for a woman, or less than 1800 kcal for a man. As another example, the target BMI objective may be selected to be above a weight considered healthy for the user.

Additionally or alternatively, the system 100 may allow the user to provide feedback to set or adjust one or more of the objectives or one or more of the recommendations, or a combination thereof. For example, if the user does not desire to reduce their caloric intake by the recommended amount, the user may adjust the restriction, thereby affecting the objective.

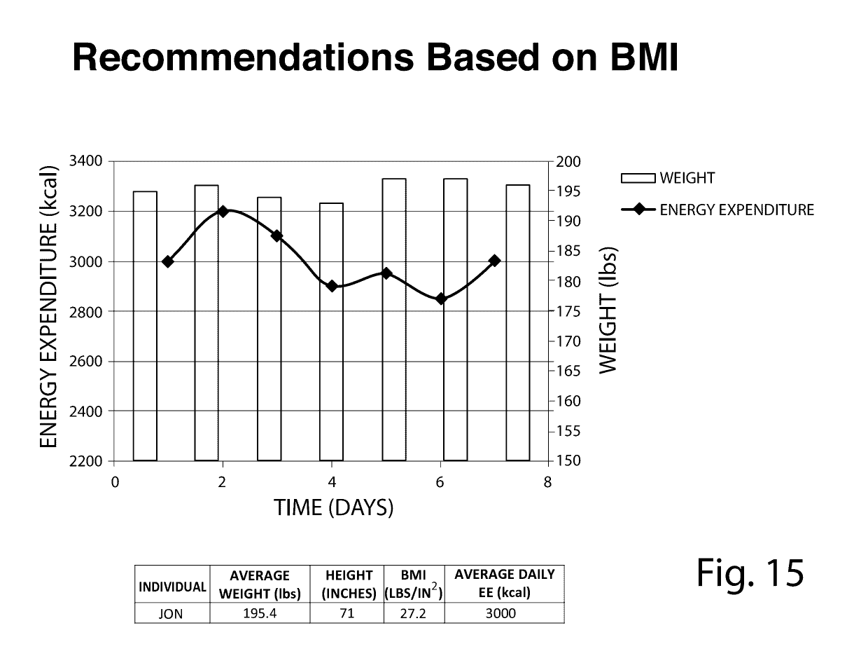

An example formulation of a caloric restriction recommendation will now be described in connection with FIG. 15. The user in this example has been determined to have an average weight of 195.4 lbs. during the initial period. Based on the tables of FIG. 14, the system may determine this weight corresponds to a BMI of 27.2 lb/in2, which is an overweight range, and may recommend that the user try for a BMI of 24.5 lb/in2, which is in the normal range. The target BMI may correspond to a target weight, such as 176 lbs, for the user, and the system may recommend that the user reduce his caloric intake by 500 kcal/day below his average daily expenditure of 3000 kcal/day.

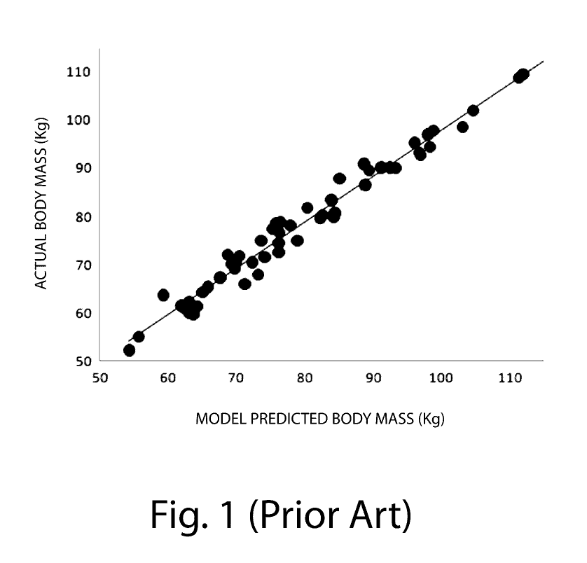

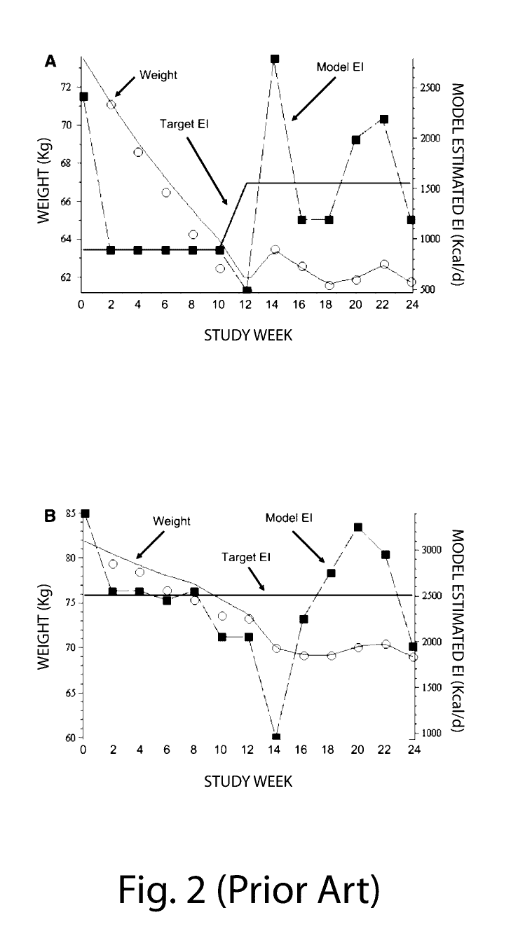

The system 100 may utilize one or more models to determine the suggested or recommended reduction in caloric intake. As an example, a model capable of predicting weight or body mass of a user based on energy intake is depicted in FIGS. 1 and 2, using the following equation (2), where FFM is fat-free mass, FM is fat mass, EI is energy expenditure, and W is weight:

$$1020 \frac{d FFM}{d t} + 9500 \frac{d FM}{d t} = EI - ( 0.075 EI + mW + \frac{s}{1 - s} ( 0.075 EI + mW + ( 1 - a ) ( {c}_{i} {W}^{n} - {y}_{i} ( {A}_{e} + \frac{1}{365} ) ) + C ) + ( 1 - a ) ( {c}_{i} {W}^{n} - {y}_{i} ( {A}_{e} + \frac{1}{365} ) ) )$$

Using this model and other models, characteristics, such as caloric intake and body mass, may be predicted based on one or more factors, such as caloric expenditure, weight, and body composition. The example model in FIG. 1 may enable prediction of a user's weight based on a variety of factors, including dietary induced thermogenesis (DIT), volitional physical activity (PA), resting metabolic rate (RMR), and spontaneous physical activity (SPA).

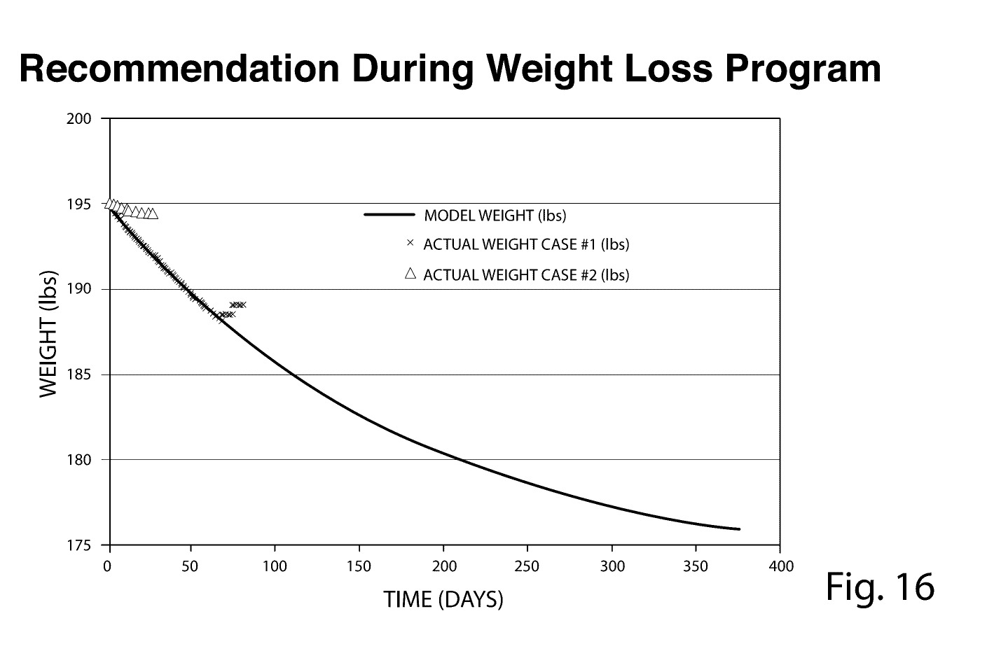

During the initial monitoring period, the system may estimate energy expenditure based on activity of the user, DIT, and RMR. The DIT may be an approximation based on an estimate of the user's caloric intake, the RMR may be approximated based on the user's characteristics such as sex, age, and weight, and the physical activity may be calculated using the accelerometer located on the personal device 10 or an equation that approximates a person's PA using their weight and a proportionality constant, or a combination of both. The weight and energy expenditure may be calculated each day and compared to a predetermined standard deviation limit and number of days. For example, if the number of days in the initial period is 3 days and a standard deviation is chosen as 1 kg for weight and 100 kcals for energy expenditure, the user in the monitoring phase may be considered stable and ready to progress to the diet if their weight fluctuated less than 1 kg in 3 consecutive days and their energy expenditure fluctuated less than 100 kcals in 3 consecutive days. The system may then take the averages of the 3 weights and the 3 energy expenditures to get a starting weight and EE. The model may assume an individual entering into a weight loss program is weight stable—e.g., not gaining or losing weight. And, the model may assume that all or nearly all of the caloric difference, energy stored (ES) (the difference between energy intake (EI) and energy expended (EE)) is originating from reduced caloric intake and not an increase in overall energy expenditure. By assuming the EE is generally equivalent to the EI, the system 100 may iteratively reduce the EI in the model of FIGS. 1 and 2 until the target weight loss is achieved for a target period. As shown in FIG. 16, the model may be utilized to develop a predicted weight for the user over time. The predicted weight loss shown in FIG. 16 is computed based on the model described in connection with FIGS. 1 and 2. Once the EI for the target weight loss is calculated, the system 100 may provide a corresponding recommendation to the user. As in the example recommendation outlined above, the recommendation may include a caloric restriction of 500 kcal/day to achieve the target weight loss. As another example, characteristics of the user, such as energy intake and body mass, may be predicted according to the methods described in the article titled, “A simple model predicting individual weight change in humans”, published Jul. 27, 2011, to Diana M. Thomas et al., in the Journal of Biological Dynamics and the article titled, “A computational model to determine energy intake during weight loss”, published in Oct. 20, 2010, to Diana M. Thomas et al., in the American Journal of Clinical Nutrition—the disclosures of which are hereby incorporated by reference in their entirety. At this stage, the user may attempt to adhere to the one or more recommendations.

While the user tries to follow the plan, the system 100 may continue to track characteristics of the user to determine user adherence to the one or more recommendations. Steps 314 and 316. For example, the user may continue to automatically provide their daily weight via the scale 110. The user may or may not continue to wear the personal device 10. If the user does not wear the personal device 10, the scale 110 may enable the user to provide daily weight to the system 100. In one embodiment, the personal device 10 may track energy expenditure in addition to daily weight in conjunction with the scale 110. Additional factors or characteristics related to the user may also be monitored and tracked, as described herein, including body composition. In one embodiment, the system 100 may analyze the tracked information using one or more models to determine adherence to the one or more recommendations. For example, the one or more models may provide predictions about the user based on monitored factors, such as weight and energy expenditure. Using these predictions, the model may aid in determining if the user is on track to achieve a target goal, such as target weight loss. In this way, the system 100 may determine adherence without using energy intake information manually entered by the user, and avoid associated user error or deception. For example, as shown in FIG. 16, weight measurements for two individuals are shown in conjunction with the same predicted weight loss model—a 500 kcal/day caloric restriction for 365 days. As can be seen, the weight of one user deviates from the model in the early stages of the program, while the weight of the other user tracks the model in the early stages but begins to deviate later on at about 70 days. The deviations can cause the system to interject a recommendation, which can include a change in the model, a change in the dietary regimen, and/or a change in the exercise regimen.

Deviations and their associated timing may be indicative of various factors. For example, deviations in the early stages may be indicative of a user's lack of adherence to the one or more recommendations. Alternatively, a deviation in the early stages of the plan may indicate the recommendation for an individual may have been incorrect from the start such that their actual weight does not follow the predicted weight loss model. In this case, the system may provide a recommendation, and potentially reevaluate the model for the individual. A deviation in the later stages of the plan may indicate the recommendation for the individual was correct from the start but that the individual stopped following the recommendation. Alternatively, deviations in the later stages may be indicative of a user's adherence to the one or more recommendations but that other factors have affected the user's progression. Whether an individual has stopped following the recommendation may be determined based on a variety of factors, such as timing and the extent to which the deviation occurs from the predicted mode. An example determination may include calculating an X-bar chart, which is used to determine the reproducibility of manufacturing processes. In this calculation, there is a mean value calculated from multiple samples, where the samples vary around the mean value by some determined threshold. In an embodiment according to the present invention, the samples may correspond to the user's weight. As long as the user's weight samples vary around the predicted model within the threshold, the system may recognize that the user is adhering to the diet. However, if a weight sample or value exceeds the threshold, the system may recognize this deviation as an indicator that the user is not adhering to the diet. Additional analysis and rule sets may be implemented as well to capture and recognize scenarios where the person may not be adhering to the diet, but remain under the threshold. For example, the system may recognize that three consecutive points larger than the expected value but still less than the threshold may be indication the user is trending away from the prescribed plan and potentially respond accordingly.

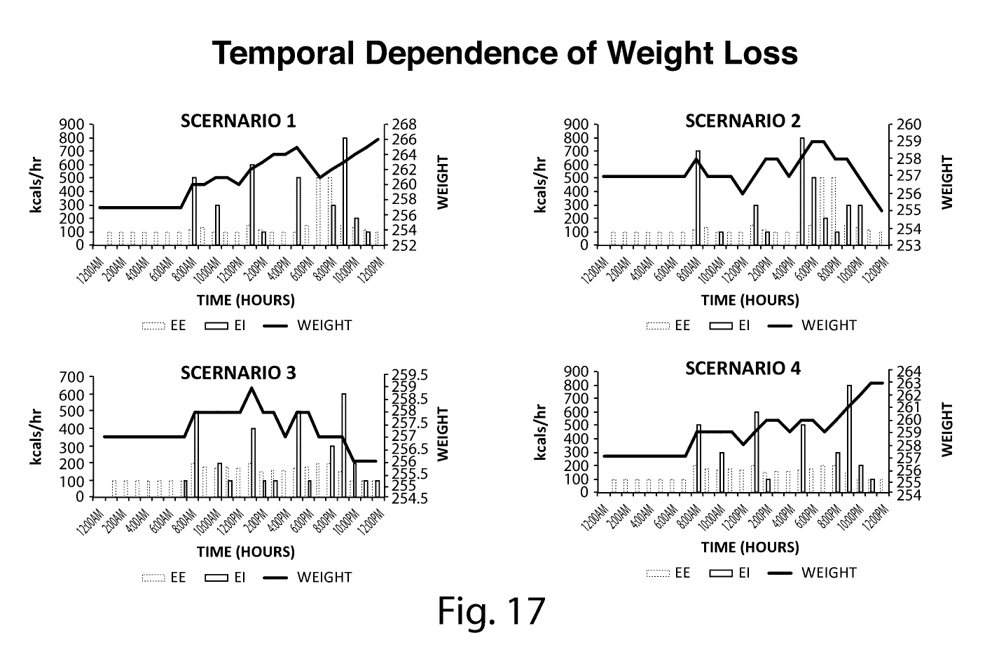

If it is determined that a deviation from the predicted model is not the result of a lack of adherence to the recommendations, the system 100 may further analyze information related to the user to attempt to account for the deviations. In one embodiment, the system may determine that the distribution of energy expenditure and energy intake over a time interval has an effect on the user's ability to track the predicted model. To account for this distribution, the system 100 may request or obtain information about when and how much the user intakes energy. As shown in FIG. 17, a 257 lb. individual consuming 3400 kcal and burning 3470 kcal over one day may burn energy in different ways (four ways are shown), depending on the timing between energy intake and energy expenditure. That is, each scenario represents a day where this person burns and eats the same amount of calories in 4 different ways. The system 100 may recommend timing for energy intake and expenditure to the individual to improve their ability to meet the target weight based on a database or a table of information. Alternatively, the system may monitor the user to determine a more efficient or optimal ratio and timing between energy intake and energy expenditure to achieve a target weight. The monitored information used as a basis for this determination may be historical data tracked in accordance with an embodiment of the present invention, or may be initiated going forward based on a determination that the user's progress has deviated from the predicted model. By optimizing the ratio between caloric intake and expenditure over time the system can recommend to an individual how they can more efficiently adhere to their program. By tracking historical data, the system can recommend which scenario works best for an individual.

In one embodiment, a dynamic version of the model depicted in FIGS. 1 and 2 may be adjusted based on a calculated EE. This may be accomplished by tracking an individual's energy expenditure using one of a variety of methods and tracking weight and/or body composition using one of a variety of methods. After the initial period, the system may determine that EE for the user has shifted from the EE monitored and used in developing the one or more recommendations with the model in the initial period. Because the EE for the model is assumed to be substantially similar to the EI, the change or shift in that EE for the user may affect the predictions developed in the initial model. If the measured weight is tracking with the predicted weight or below, nothing may be done. This may suggest that the person is exercising more and eating the same such that they are increasing their rate of weight loss. As long as the proportion of the user's weight loss is not largely from a loss in FFM, and the user has not dipped below a healthy BMI, then nothing may be done, or no recommendation may be given. However, if a large proportion of weight loss is associated with a loss in FFM, or the user dips below a healthy BMI, the system may provide a recommendation to attempt to correct the situation. Accordingly, if the user's weight is determined to be higher than the predicted weight by the X-bar rules, the system may initiate the evaluation of the model using an updated EE for the user to account for the corresponding shift from the initial EE. In this way, recommendations, such as a caloric restriction recommendation, may be adjusted based on changes in the user's behavior or activity level. The system may handle changes in a user's behavior or activity level in one or more ways. For example, if at time t, the user's weight violates one of the predetermined rules for adherence, and the user's weight is higher than predicted, the previous X-chart values of EE may be averaged together to get a new EE at time t. This new EE may be compared to the baseline EE; if they are the same, the system may recalculate the EI in the model of FIGS. 1 and 2 based on the averaged EE at time t and the corresponding weight at time t. Based on this determination, the system may indicate to the user how much they may have over ate in order to reach that weight. If the same scenario occurred, but the user's new EE is less than the baseline EE, a similar modeling process may be performed to determine if the user's weight increased due to the lower, new EE, or if the user also over ate. The system may monitor motion and activity of the user, which may be used to determine how the user reached a particular that which is not adhering to the prescribed model. As mentioned herein, this monitoring may be conducted continuously, intermittently, periodically, or based on the occurrence of an event.

Based the determination of whether the user is adhering to the one or more recommendations, the system may provide feedback to the user. Steps 316, 318, 320. For example, if it is determined the user's energy intake or weight is larger than the target energy intake or target weight based on the caloric restriction recommendation, the system 100 may provide feedback to the user recommending a change or providing a suggestion, such as to reduce caloric intake further or to increase energy expenditure. In one embodiment, one or more devices in the system may communicate with each other to provide suggestions to the user, including, for example, a suggested food recipe, or a replacement item for a food recipe, or a food or dietary supplement, or a combination thereof. On the other hand, if it is determined the user's energy intake is on track with the target energy intake based on the caloric restriction recommendation, the system 100 may provide positive feedback to the user to maintain their current plan. The determination of whether the user adheres to one or more recommendations may be conducted continuously, intermittently, periodically or based on the occurrence of an event, such as a perceived deviation from the weight loss program.

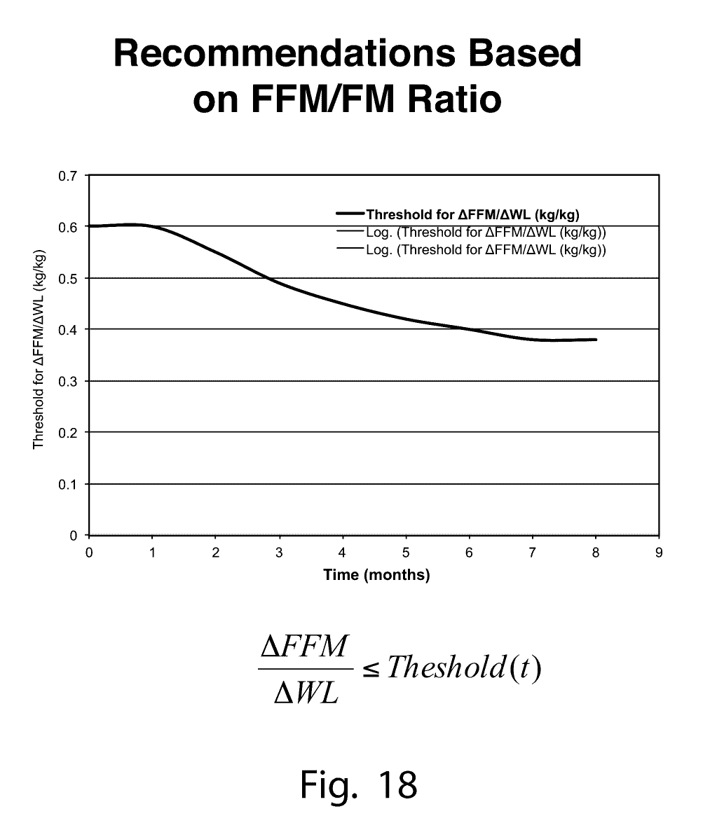

In one embodiment, the system 100 may provide a recommendation based on a determination that the progression of weight loss associated with a user includes a loss of FFM considered excessive or to exceed a threshold. In this way, the system 100 may try to ensure the user maintains a healthy ratio of FFM to FM. As shown in FIG. 18, the system may calculate a threshold ratio between loss of FFM and weight loss. The plot shows an example of what may be considered healthy weight loss of FFM as a fraction of total weight loss (WL) over time on a diet. This healthy ratio may be used to set a maximum threshold for the faction of weight loss that can occur as FFM. If the system 100 determines that an individual is losing too much FFM using the equation shown, it may provide a recommendation accordingly, such as to increase protein intake to overcome the loss in FFM.

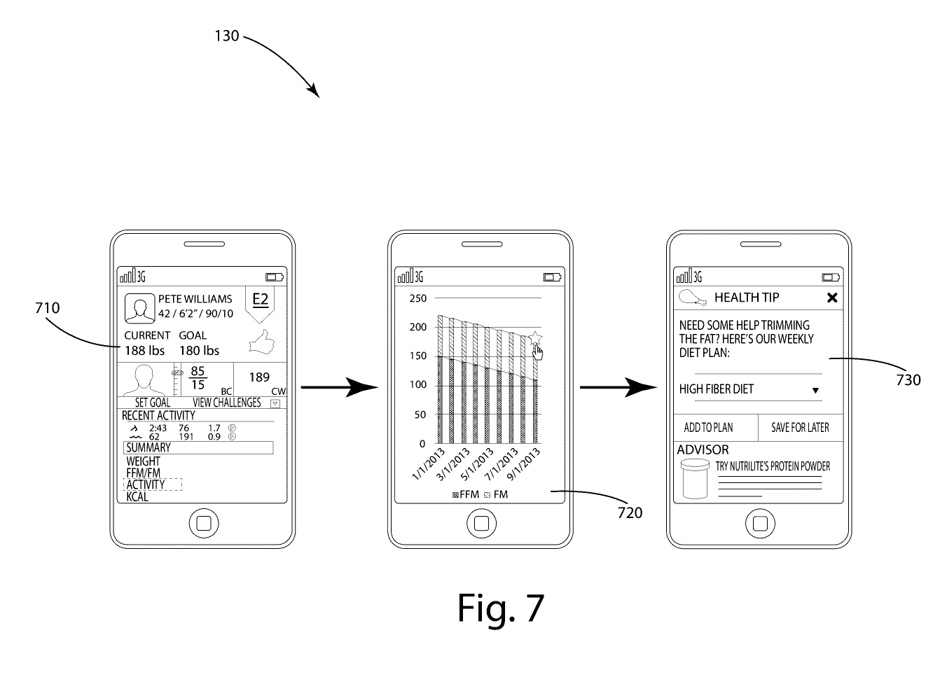

FIG. 7 includes illustrations of examples of mobile interfaces for displaying data to a user and the levels to which a user can interact with their data. The panel on the left is an overall user dashboard. The panel in the middle is a representation of user weight and body composition (FM and FFM). This middle panel is realized when the user selects weight on the dashboard. The panel on the right is realized when a user is prompted to click on the data (shown as a star). Based on trends in the data, the system recommends an action. In this example, the system realized the user was losing weight, but this weight loss was attributed to FFM and not FM so the system recommends that the user try protein powder.

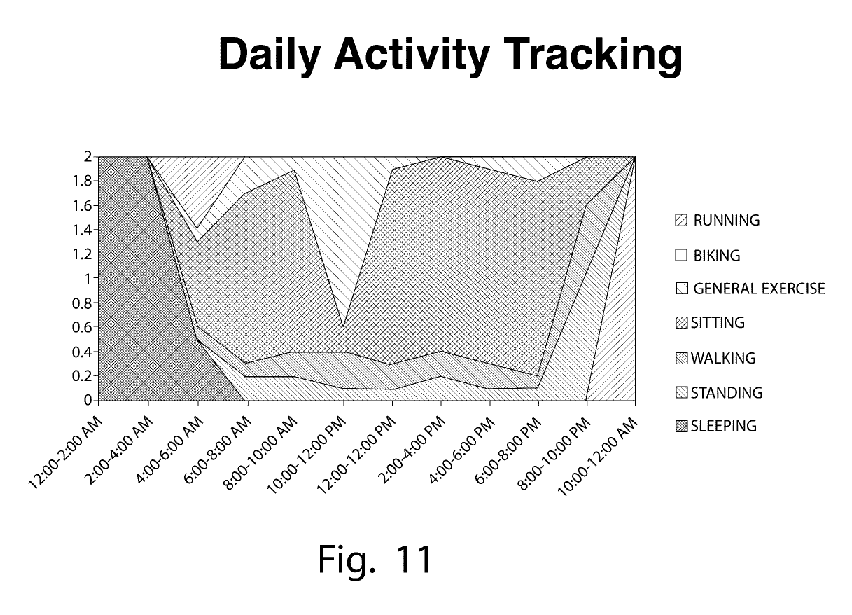

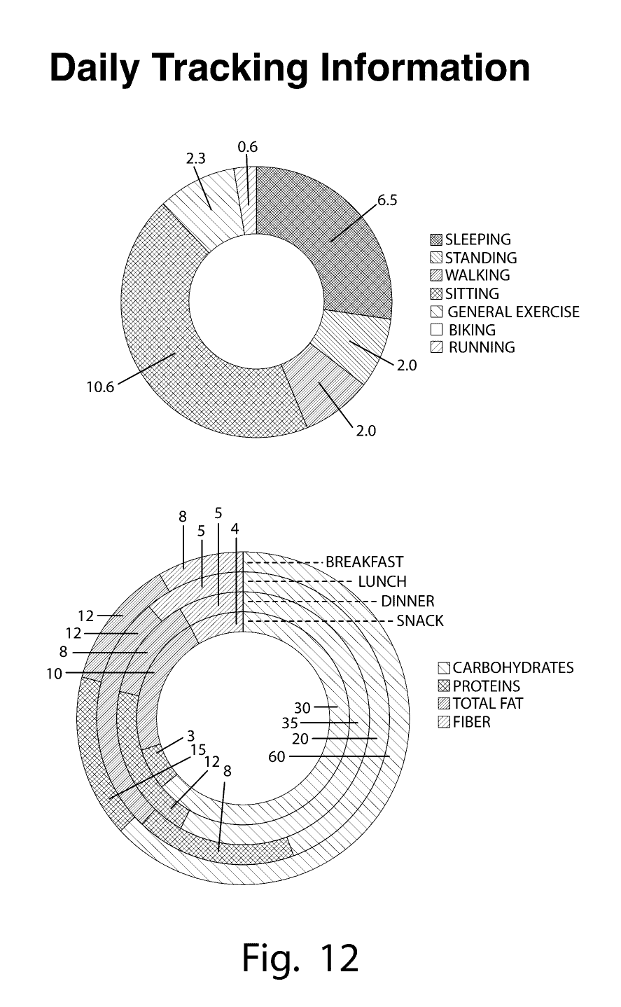

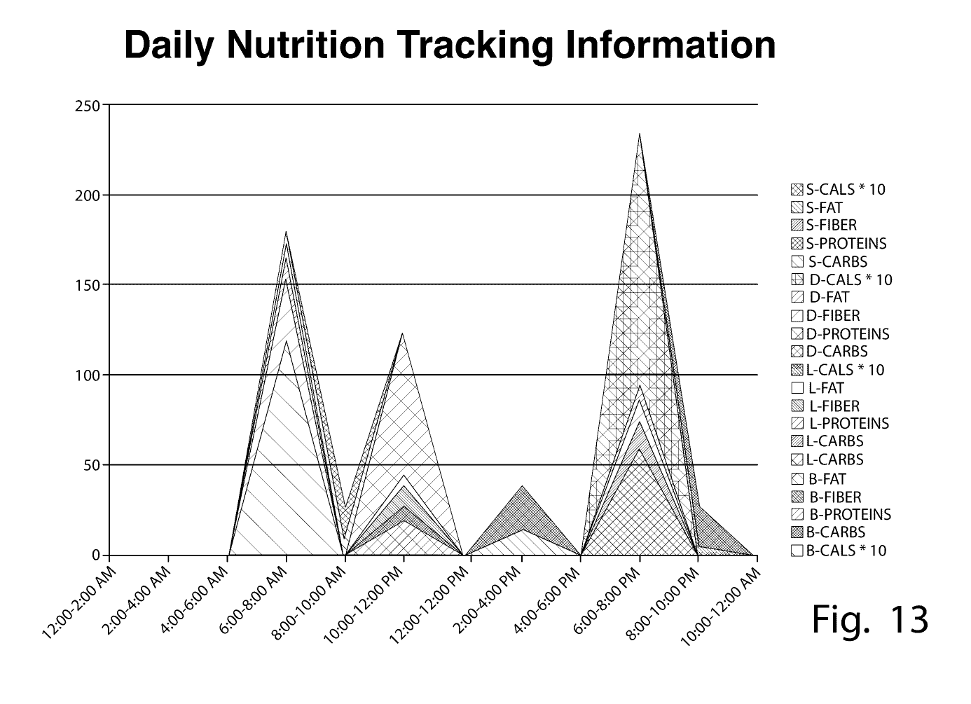

As shown in the illustrated embodiments of FIGS. 6 and 7, the system 100 may interact with the remote device 130 to provide feedback and recommendations to the user, including providing recommendations in accordance with the method 300. For example, the remote device 130 may include a user interface 610, 710 or dashboard that enables the user to track their activity levels and recommendations in a useful and interactive manner. Areas of the user interface may activate further views to aid the user in understanding their information and recommendations. The user interface available on the remote device 130 may include information about the user such as a breakdown of the user's activity for the day 620, 630, including for example energy expended while running, standing, sitting, or being seated. The user interface may indicate to the user energy expended during their activities using metaphorical comparisons 640 to other activities, such as eating a quarter cheeseburger, performing 100 push-ups, or losing 1/100 in pants size. The user interface may also enable the user to view their monitored body composition 720, including viewing a comparison between FFM and FM, to aid the user in achieving adherence to the one or more objectives. The user interface may also provide supplemental recommendations 730 that may aid achieving adherence to the predicted model or the one or more objectives. For example, if it is determined the user is losing FFM rather than FM or other trends, the user interface may provide a suggestion area, depicted as a star, that may activate a suggestion, such as to try a protein powder. The user interface 610, 710 may also provide information related to the user's activities, including a daily activity log similar to the log shown in FIG. 11, which shows the relative amount of time spent performing an activity throughout the day. For example, between 8-10 a.m., the daily activity log indicates the user spends a greater amount of time sitting than walking or standing over a period of two or more days. As shown in FIG. 12, the daily activity log may provide similar information but using a pie chart instead. The daily activity log may also break down the distribution of food intake based on times of the day, such as breakfast, lunch, dinner, and snack times. If the user understands these interactions they can look back on historical data and optimize the ratio of intake and expenditure to best adhere to the prescribed health management program. FIG. 13 illustrates yet another manner of conveying and analyzing the user's food intake and source of nutrition in relation to times of the day. By understanding this ratio and how energy expenditure interacts with this, the user can better optimize their health management program.

A method of tracking user adherence to one or more objectives will now be described with respect to the illustrated embodiment of FIG. 3B. As shown, the method, designated 400, is similar to the method 300 described in connection with FIG. 3A with some exceptions. The method 400 may be implemented in a system to track information related to or characteristics of a user to develop a user profile, and, based on the tracked data, form one or more health metrics or objectives or monitor adherence to one or more objectives, or a combination thereof. The method 400 may also enable the user to interact with a system according to an embodiment described herein to provide feedback to the user. In one embodiment, the feedback may include a recommended caloric restriction to achieve adherence to the one or more objectives, similar to the method described above with respect to the illustrated embodiment of FIG. 3B.

Starting with step 410, the user may initiate a health management program according to the method 400 within the framework of a system 100, including a personal device 10. Although described in connection with the system 100 described in connection with FIG. 5, it should be understood that the method 400, or one or more steps thereof, may be implemented in any system or component described herein. For an initial period of time (e.g., a week), the weight of the user is determined on a periodic basis, such as on a daily basis, the user wears the personal device 10. The weight of the user may be determined using a scale, such as the scale 110, which automatically reports the user's weight to a component within the system 100, such as the personal device 10 or the external server 150. Alternatively, the user may manually enter their weight into the system.

The system 100 may track a variety of characteristics or obtain information related to the user during the initial period, including tracking one or more of energy expenditure, blood pressure, hydration, resting heart rate, stress, and sleep. The personal device 10, as outlined above, may include one or more sensors capable of tracking this information. For example, a determination of energy expenditure, sleep, and heart rate may be based on accelerometer information obtained while the user wears the personal device 10 for the initial period. The system 100 may also include a blood pressure measurements device, such as a blood pressure cuff, having wireless communication capabilities such that it can communicate wirelessly with other devices in the system 100, such as the personal device 10. Information obtained during the initial period may also include family history, or DNA analysis, indicative of potential medical issues or a predisposition toward medical conditions, such as high blood pressure.

Based on information related to the user, the personal device 10 may determine one or more of average daily energy expenditure, average resting heart rate, average blood pressure, average FM, average FFM, and average hydration level. These parameters may be used as a basis for developing a plan or one or more objectives for the user. It should be understood that the method 400 may develop a plan or one or more objectives based on any type of information related to the user, and is not limited or tied to developing a plan based on all or a subset of the parameters outlined herein. The data collected during the initial period may aid the system 100 in generating one or more objectives for the user that are likely to achieve user adherence. Step 412. The one or more objectives may include a healthy weight, or healthy weight loss, a target BMI, a target body composition, or a target blood pressure, or a combination thereof.

In the illustrated embodiment of FIG. 3B, the method 400 may utilize a model, such as the model described above in connection with the method 300, to generate one or more recommendations to achieve the objectives. Step 412. For example, the system 100 may recommend a caloric restriction to achieve an overall healthy state and a target weight loss. The system 100 may also provide one or more recommendations to achieve other objectives, including those outlined above such as a target blood pressure. For example, the system 100 may suggest an exercise regimen or a low sodium diet to achieve a healthy blood pressure in conjunction with the target weight loss. As another example, the system 100, may suggest an exercise regimen to achieve a lower target resting heart rate. In yet another example, the system 100 may recommend drinking water to increase hydration levels toward a target.

While the user tries to follow the plan and objectives laid out according to the method 400, the system 100 may continue to track characteristics of the user to determine user adherence to the one or more recommendations. Steps 414 and 416. For example, similar to the method 300, the user may continue to automatically provide weight information utilizing the scale 110. The system 100 may also track one or more additional factors related to or characteristics of the user, such as energy expenditure, body composition, hydration, blood pressure, resting heart rate, stress levels, and sleep. The system 100 may analyze the tracked information using one or more models, such as the model described herein with respect to method 300, to determine adherence to the one or more recommendations. Step 416. If the system 100 determines the user is on track to achieve a target objective, such as target weight loss, the system 100 may inform the user to continue with their current program. Step 420. If the system, however, determines the user has deviated from the recommendations based on a comparison between the prediction model and the recommendations, the system 100 may provide further recommendations to the user. 418. For example, if one or more of the user's daily weight, changes in body composition, changes in hydration levels, changes in blood pressure, changes or increases in sodium levels indicated by sweat, stress levels, and sleep levels indicate the user has deviated from the recommendations, the system may inform the user accordingly, and may provide a recommendation to help achieve adherence to the objectives. As mentioned above, it is possible the user has followed the recommendations but has still deviated from the predicted model. If the system 100 determines this has occurred, a recommendation or further analysis may be conducted or suggested, similar to the method 300.

As noted above, the present invention may be part of a larger system (or network) of products that is intended to assist a user in enhancing health and well-being (generally referred to as a health and wellness network). To facilitate this enhanced functionality, the health and wellness network may include various networked health and wellness devices that collect and store a variety of types of information about the user and the user's activities, such as weight, body composition, heart rate, blood pressure, hydration, diet, exercise, sleep patterns, nutritional intake and other factors that may be relevant to health and well-being. The health and wellness network may then be able to assist the user in maintaining a high level of health and well-being by processing the collected information and providing the user with recommendations for maintaining or improving health and well-being. Health and wellness networks, as well as various health and wellness devices, are described in U.S. Provisional Application No. 61/567,692, entitled Behavior Tracking and Modification System, filed Dec. 7, 2011, by Baarman et al; International Publication No. WO 2013/086363, entitled Behavior Tracking and Modification System, filed Dec. 7, 2012, by Baarman et al; U.S. application Ser. No. 13/455,634, entitled Pill Dispenser, filed Apr. 25, 2012, by Baarman et al; and U.S. application Ser. No. 13/344,914, entitled Health Monitoring System, filed Jan. 6, 2012, by Baarman et al, all of which are incorporated herein by reference in their entirety.

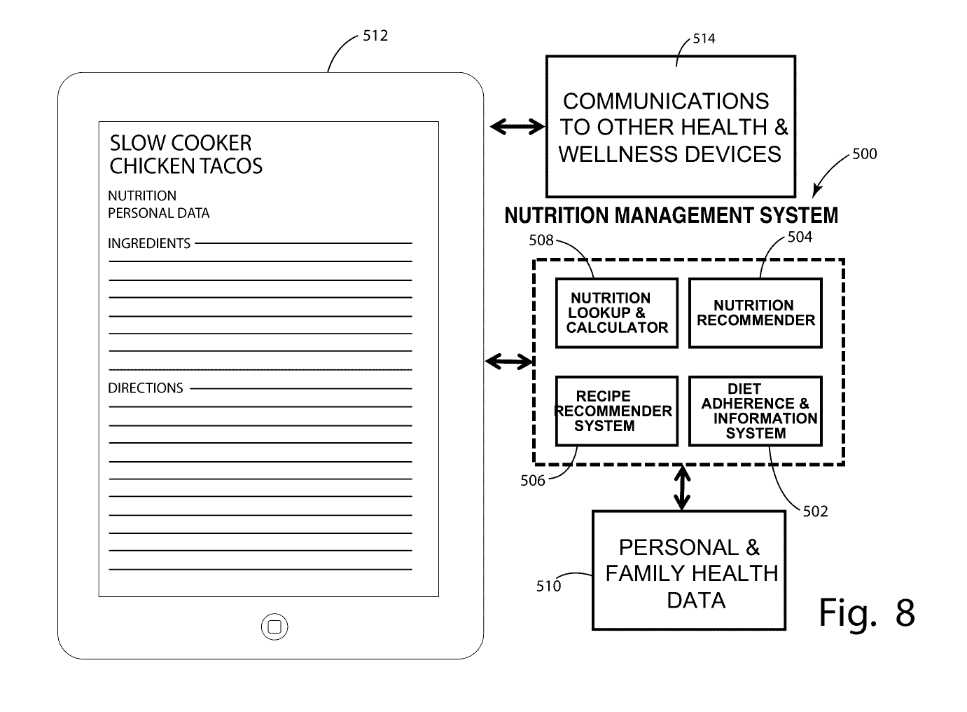

The system of the present invention may be integrated into the health and wellness network in a variety of different ways. For example, the information collected and recommendations provided by the system of the present invention may be used by other systems within the network. In one embodiment, the system of the present invention may be part of a nutrition management system that is implemented within the health assistance network. The nutrition management system may be configured to provide the user with nutrition-related recommendations, such as general nutrition recommendations and/or specific recipe recommendations. Referring now to FIG. 8, the nutrition management system 500 of one embodiment may include the diet adherence system of the present invention 502, a nutrition recommender 504, a recipe recommender system 506 and a nutrition lookup and calculator 508. The nutrition management system 500 may communicate with a network device or database 510 that includes personal and family health data. In this embodiment, the diet adherence system 502 provides input to the nutrition management system 500. More specifically, in use, the nutrition management system 500 may be configured to make nutrition recommendations and recipe recommendations that take into account the weight loss or body composition objectives of the user as provided by the diet adherence system 502, as well as the health and wellness information collected or otherwise obtained by the diet adherence system 502.

In the embodiment of FIG. 8, the health and wellness network communicates with the user via an application running on a personal electronic device, such as a tablet computer 512. The application running on the tablet computer 512 may be capable of interacting with nutrition management system 500 and other health and wellness devices 514 included in the network. In the embodiment of FIG. 8, the nutrition management system 500 may collect information directly from the diet adherence system 502 and the network database 510, and may collect information indirectly from other networked devices 514, for example, via the tablet computer 512. In operation, the nutrition recommender 504 analyzes the information collected from the diet adherence system 500, the network database 510 and any other networked devices 514 to develop a nutrition recommendation for the user. The nutrition recommendation will be formulated to help the user stay on track with the user's goals and objectives and to generally enhance health and wellness. The recipe recommender system 506 of this embodiment is configured to make recipe recommendations that help to implement the nutrition recommendations for the user. The recipe recommender system 506 may interact with the nutrition lookup and calculator 508 when developing recipe recommendations. The nutrition lookup and calculator 508 may include nutrition information for various ingredients. For example, the nutrition lookup and calculator 508 may include a database that contains the nutritional content of food ingredients based on weight. In use, the nutrition lookup and calculator 508 may provide the recipe recommender system 506 with nutrition information for select recipes, thereby allowing the recipe recommender system 506 to provide appropriate recipe recommendations that are aligned with the nutrition recommendations for the user. In addition to providing recommendations relating to weight loss and body composition, the nutrition management system 500 may also provide a recommendation relating to other health factors, such as recommending recipes for a low sodium diet when blood pressure is a concern or recommending low fat and low cholesterol recipes when cholesterol level is a concern.

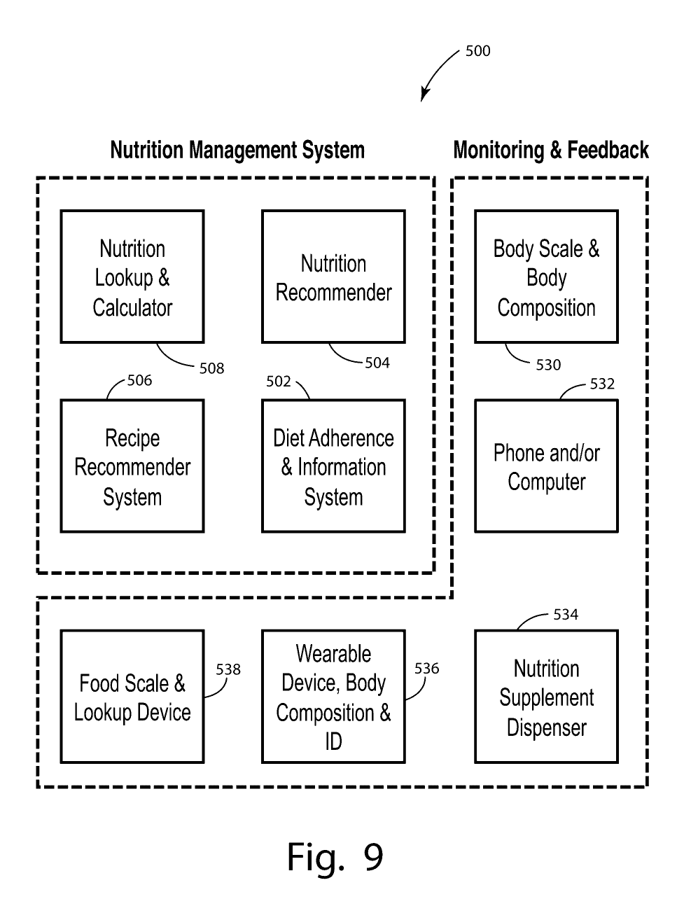

The health and wellness network shown in FIG. 8 is merely exemplary. The nutrition management system 500 may be incorporated into a variety of different health and wellness networks, and may be capable of interacting with a variety of different health and wellness devices. For example, FIG. 9 is a block diagram showing a variety of health and wellness devices that might communicate with the nutrition management system 500. As shown, the devices may include a body scale and body composition device 530, a phone and/or computer 532, a nutrition supplement dispenser 534, a wearable device 536 (e.g., personal device 10) and a food scale and lookup device 538. These devices may communicate wirelessly or via wired communications. In the illustrated embodiment, the devices communicate wirelessly using a conventional wireless communication protocol, such as Bluetooth or WiFi. In this embodiment, the body scale and body composition device 532 may be a conventional communication-enabled scale that takes body weight measurements and body composition measurements. For example, the body composition measurements may be BMI measurements computed using measured weight and height information provided by user or may be measurements of the ratio of FM/FFM using bio-impedance sensors. The phone and/or computer 532 may be incorporated into this exemplary network to provide a user interface for exchanging information with the user. For example, the phone and/or computer 532 may run an application configured to interact with the other devices in the health and wellness network. The application may be configured to collect any desired information from the user and to provide the user with access to information and recommendations. The nutrition supplement dispenser 534 may be configured to dispense nutritional supplements determined to be appropriate by the nutrition management system 500 or by some other network devices tasked with that function. For example, the nutrition supplement dispenser 534 may itself be configured to determine appropriate supplements based on information collected and maintained within the health and wellness network. As described in more detail above, the wearable device 536 may be worn by the user and may include various sensors intended to collect information about the user's physical activities and health characteristics, such as body composition. The wearable device 536 may be provided with essentially any sensors that may be useful for the system. For example, the wearable device 536 may include a bio-impedance sensor, a heart rate monitor and/or a sweat sensor. The food scale and lookup device 538 may be provided to allow accurate input of food consumption information. For example, the food scale and lookup device 538 may allow a user to measure food that is going to be consumed. The device 538 can also provide additional functionality by looking up nutritional information for the weighed foods. The device 538 can then provide the nutrition management system 500 (and other network devices) with weight and nutritional information for consumed foods.

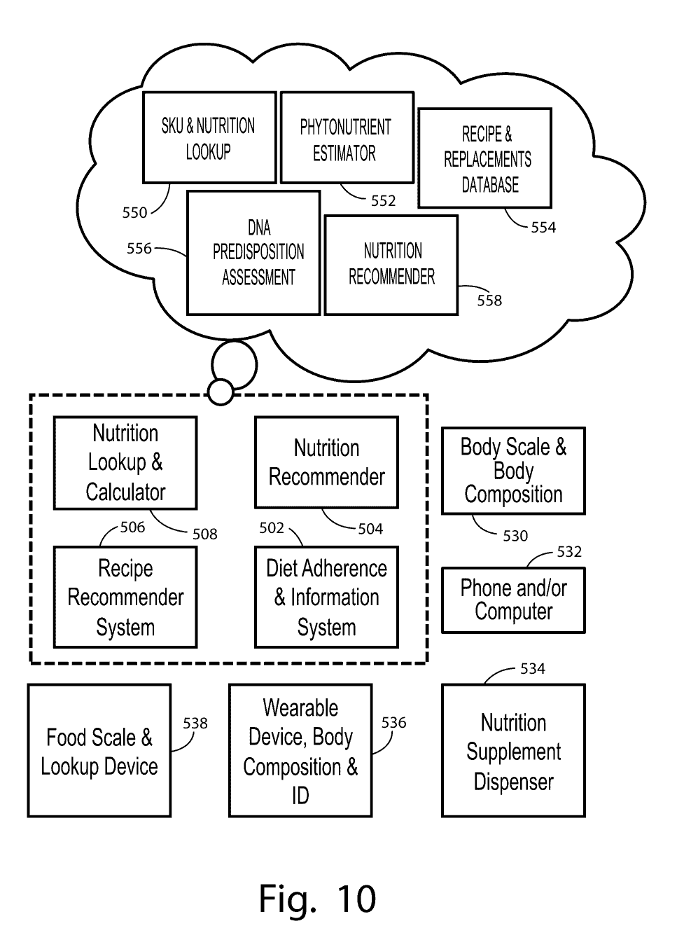

As noted above, the health and wellness network may be implemented with a web-based cloud. As shown in FIG. 10, the nutrition management system 500 and various network devices of FIG. 9 can be interconnected using a wireless networking technology that utilizes internet-based communications. The various network components may be connected to the internet via wireless or wired connections. For example, the devices may connect to the internet using a standard wireless communication protocol, such as through the use of a WiFi router and WiFi communications, or a wired communication protocol, such as through the use of wired connections to an Ethernet switch. Although the web-based health and wellness network may include essentially any combination of devices, the embodiment of FIG. 10 includes a cloud-based environment in which the nutrition management system 500 has access to a SKU and nutrition lookup device 550, a phytonutrient estimator 552, a recipe and replacements database 554, a DNA predisposition assessment device 556 and a nutrition recommender 558. In this embodiment, the SKU and nutrition lookup device 550 may be capable of obtaining SKU information and looking up nutrition information for the product identified by the SKU. The device 550 may obtain the nutrition information from a table or other collection of data that associates nutrition information with products by SKU. The SKU and nutrition lookup device 550 may have an integrated scanner, such as a barcode scanner, to obtain a product's SKU. The nutrition database may be resident in memory of the device 550 or it may be in a separate device, such as a network database (not shown). The phytonutrient estimator 52 of this embodiment is configured to provide phytonutrient information for specific plants based on weight or volume. The phytonutrient estimator 52 may be used in determining the phytonutrient content of consumed foods or in estimating the phytonutrient content that may be contained in recommended foods. The recipe and replacements database 554 may be a database containing a collection of recipes, as well as substitute ingredients that might be useful in following a specific diet regimen. For example, the database may provide substitute ingredients that provide a low-sodium recipe or a low-fat recipe. This database 554 may provide data to the recipe recommender system 506. For example, the recipe recommender system 506 may interact with the recipe and recommender database 554 each time that it makes a recommendation. As another example, the recipe recommender system 506 may maintain an internal database of recipes and replacements, and it may periodically update that database with recipes from the recipe and recommender database 554. In this embodiment, the DNA predisposition assessment device 556 is configured to assess a user's DNA predisposition and make recommendations intended to address those predispositions. For example, the device 556 may assess family history of heart disease and may recommend actions that could help the user lower blood pressure or cholesterol. For example, the system may recommend an exercise regimen and/or a diet that is low in fat or low in cholesterol. The DNA predisposition assessment device 556 may also provide recommendations based on actual DNA sequencing. For example, the user may provide a DNA sample and analysis of the DNA may be performed to determine genetic predisposition. The result of the DNA analysis may be stored in the DNA predisposition assessment device 556 and made available to other devices in the health and wellness network. The system may also recommend that a user see a doctor if recommended actions do not have the desired effect. The cloud-based nutrition recommender 558 of this embodiment may be redundant or may provide capabilities that vary when compared to the nutrition recommender 504 incorporated into the nutrition management system 500. For example, the cloud-based nutrition recommender 558 may be configured to provide nutrition recommendations based on a larger set of data made available by a larger number of network devices.

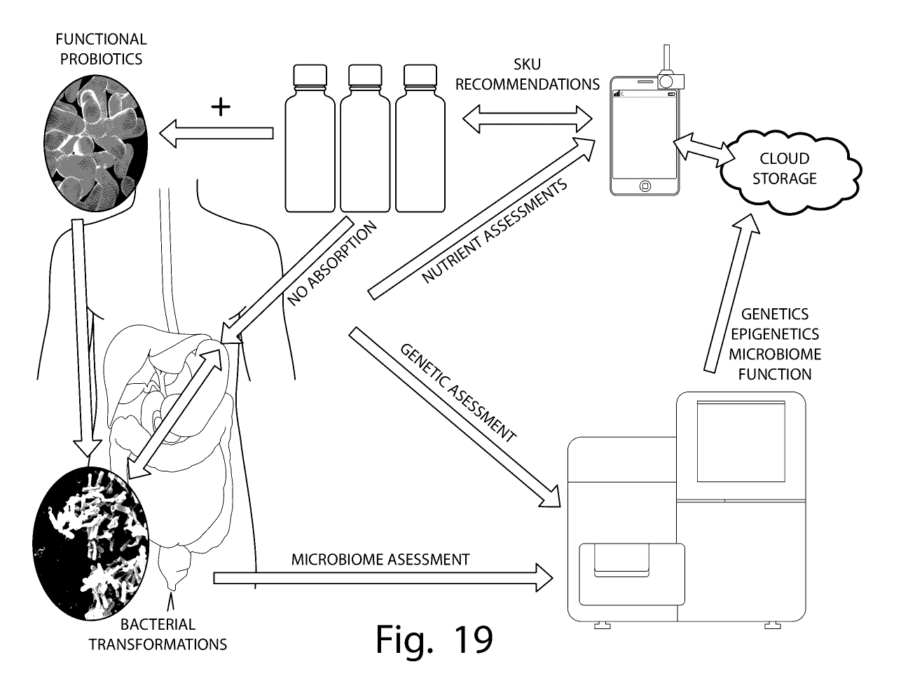

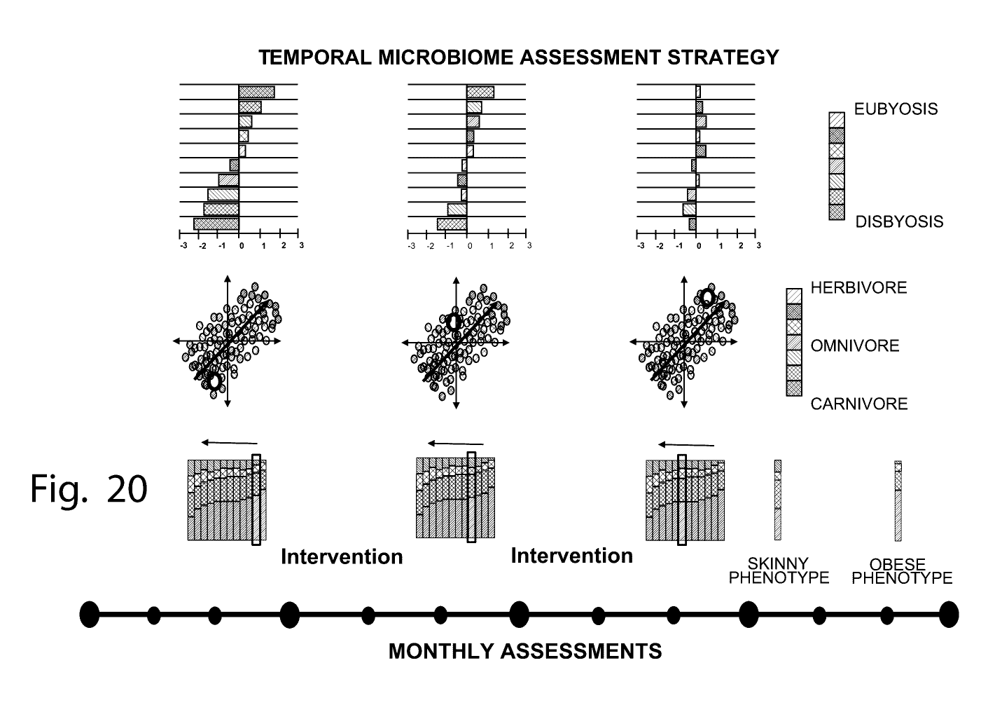

The system 100 of the present invention can additionally factor in microbiomes and genetics when managing the dietary regimen as part of an overall health program. As shown in FIG. 19 for example, microbiomes within the human body and certain genetic predispositions can impact an individual's metabolism and immune system functions. The system 100 can factor in a microbiome assessment and a genetic assessment when determining either a) the dietary regimen most appropriate for the selected weight loss program or b) the modification most appropriate for the individual at various points in the selected weight loss program. The determination can optionally be performed in a cloud server as shown in FIG. 19, the output being a suggested nutritional supplement (including probiotics), meal, meal plan, or recipe, optionally by SKU. FIG. 20 includes an exemplary temporal microbiome assessment strategy to quantify shifts in an individual's microbiomes. Bacterial communities in the intestine are shown to quickly respond to shifts in diet and activity and other perturbations in the microbiome community. Evaluating imbalances or disbyosis in the intestines can provide a responsive indicator of behavior for the user. Consequently, the dietary regimen and its subsequent modification can be more appropriately tailored to assist the individual in meeting his or her health goals.

The system 100 of the present invention can additionally monitor the bio-availability of bionutrients when managing the dietary regimen as part of an overall health program. The system 100 can factor in the bio-availability of bionutrients when determining either a) the dietary regimen most appropriate for the selected weight loss program or b) the modification most appropriate for the individual at various points in the selected weight loss program. For example, it is known that the bioavailability of certain phytonutrients and/or their metabolites can be dictated by the absence or presence of different strains of bacteria that line the gastrointestinal track. The isoflavone daidzian, for example, is commonly found in soybean plants and can only be converted to the active metabolite s-equol in individuals that have a specific composition of bacteria containing eubacterim ramulus. In addition, the ratio of the bacteria frimicutes and bacteroidets has been shown to correlate with an obese phenotype or lean phenotype. With knowledge of a) the presence or absence of eubacterim ramulus and b) the ratio of frimicutes to bacteroidets, a dietary regimen can be selected or modified to enhance the user's participation in the overall health program. For example, the system 100 can recommend a dietary regimen rich in daidzian for program participants having appropriate levels of eubacterim ramulus. For other participants, the system 100 can recommend a dietary regimen substantially free of daidzian. These considerations are equally applicable when determining modifications to the dietary regimen, and not simply when determining the dietary regiment at the outset.

To reiterate, the current embodiments can provide a method and a system for providing dietary guidance to an individual. The method can include a) receiving a selection of a health program for the individual, the health program including a dietary regimen and an exercise regimen, b) measuring the individual's caloric expenditure and/or change in body composition or body mass during the individual's participation in the health program, c) storing the measured caloric expenditure and the measured change in body composition or body mass to computer readable memory, d) determining adherence to the dietary regimen or the exercise regimen based on the measured caloric expenditure or the measured change in body composition or body mass, e) identifying a modification to the dietary regimen or the exercise regimen, and f) informing the individual of the modification. The method can further include predicting an expected change in body composition or body mass based on the health program selected by the individual and based on the individual's gender, age, height, weight, and other factors. The modification can include a change in the dietary regimen, including one or more new or modified meal plans and/or recipes having a caloric content tailored to assist the individual in meeting his or her health goals. As used above, “body composition” can include the ratio of FFM to FM or the individual's BMI. The system can generally include a first device including a first sensor to measure caloric expenditure, a second device including a second sensor adapted to measure body mass, and a computer adapted to perform the following steps based on the measured caloric expenditure and the measured body mass: a) determine an expected body mass as a function of the prescribed dietary regimen, the prescribed workout regimen, and the measured caloric expenditure, b) compare the measured body mass with the expected body mass, and c) recommend a modification of at least one of the prescribed dietary regimen and the prescribed exercise regimen based on a departure of the measured body mass from the expected body mass.

The system can include multiple devices 530, 532, 534, 536, 538 as illustrated in FIG. 9 and a nutrition management system 500 interacting with a web-based cloud 150. The nutritional management system 500 interacts with other cloud databases, allowing the individual to have his or her personal information along with the data coming from external devices along with other databases that help monitor what the individual is doing and can recommend changes to help the individual with his or her goals. They system may also be intelligent; for example, from the DNA predisposition assessment, the system could recommend that the individual lower his or her blood pressure or cholesterol in view of a family history of heart disease. Based on these answers, the system could give more weight to trying to lower blood pressure, or if it did everything it could from a health standpoint and blood pressure was still high, the system could recommend visiting a doctor to potentially obtain medication.

The above description is that of current embodiments of the invention. Various alterations and changes can be made without departing from the spirit and broader aspects of the invention. This disclosure is presented for illustrative purposes and should not be interpreted as an exhaustive description of all embodiments of the invention or to limit the scope of the claims to the specific elements illustrated or described in connection with these embodiments. For example, and without limitation, any individual element(s) of the described invention may be replaced by alternative elements that provide substantially similar functionality or otherwise provide adequate operation. This includes, for example, presently known alternative elements, such as those that might be currently known to one skilled in the art, and alternative elements that may be developed in the future, such as those that one skilled in the art might, upon development, recognize as an alternative. Further, the disclosed embodiments include a plurality of features that are described in concert and that might cooperatively provide a collection of benefits. The present invention is not limited to only those embodiments that include all of these features or that provide all of the stated benefits, except to the extent otherwise expressly set forth in the issued claims.

1. A system for providing weight loss guidance to a user, the system comprising:
a weight scale;
a caloric expenditure calculator; and
a personal device in communication with the weight scale and the caloric expenditure calculator, the personal device including a visual display and adapted to:
determine an average energy expenditure of the user based on the output of the caloric expenditure calculator,
determine a dietary regimen and a model weight loss prediction for the user based on the average energy expenditure and a user-selected target weight loss,
monitor adherence to the dietary regimen based on a comparison of the user's body mass as measured by the weight scale with the model weight loss prediction,
provide a graphical representation of the measured body mass as compared to the model weight loss prediction on the visual display,
select a modification of the dietary regimen, wherein the modification is based on a comparison of the user's body mass as measured by the weight scale and the model weight loss prediction, and
present the modification of the dietary regimen to the user using the visual display to aid the user in achieving the user-selected target weight loss.

2. The system of claim 1 wherein the modification includes a food recipe, a replacement item for a food recipe, or a food or dietary supplement.

3. The system of claim 1 wherein the personal device is a wearable device or a handheld device.

4. The system of claim 1 wherein the dietary regimen includes a caloric restriction over a prescribed time period.

5. The system of claim 1 wherein the dietary regimen includes a plurality of recipes over a prescribed time period.

6. The system of claim 1 wherein the caloric expenditure calculator is incorporated into the personal device.

7. A system for providing weight loss guidance to a user, the system comprising:
a weight scale adapted to measure body mass during the user's participation in a dietary regimen; and
a personal device including a visual display, the personal device being in communication with the weight scale over a network, the personal device being adapted to:
monitor adherence to the dietary regimen based on a comparison of the user's body mass as measured by the weight scale with a model weight loss prediction,
select a modification of the dietary regimen including a food recipe, a replacement item for a food recipe, or a food or dietary supplement, wherein the modification is based on the comparison of the user's body mass as measured by the weight scale and the model weight loss prediction, and
present the dietary modification to the user using the visual display to aid the user in achieving a user-selected target weight loss.

8. The system of claim 7 wherein the personal device is a wearable device or a handheld device.

9. The system of claim 7 wherein the personal device includes a caloric expenditure calculator that is adapted to measure energy expenditure.

10. The system of claim 9 wherein the personal device is adapted to determine the model weight loss prediction based on the user-selected target weight loss and an average energy expenditure of the user.

11. The system of claim 7 wherein the visual display is adapted to present a graphical representation of a measured body mass as compared to the model weight loss prediction.

12. The system of claim 7 wherein the personal device is adapted to access a database having nutritional data for a plurality of recipes for selecting a modification of the dietary regimen.

13. The system of claim 7 wherein the dietary regimen includes a caloric restriction over a prescribed time period.

14. The system of claim 7 wherein the dietary regimen includes a plurality of recipes over a prescribed time period.

15. A system for providing weight loss guidance to a user, the system comprising:
a weight scale;
a caloric expenditure calculator; and
a computer including a processor adapted to execute the following steps based on an output of the weight scale, an output of the caloric expenditure calculator, and a selection of a target weight loss:
determine an average energy expenditure of the user,
determine a dietary regimen and a model weight loss prediction for the user based on the target weight loss and the average energy expenditure,
determine a measured body mass at recurring intervals during the user's participation in the dietary regimen, and
provide feedback to the user including a graphical representation of the measured body mass as compared to the model weight loss prediction to aid the user in achieving the selected target weight loss.

16. The system of claim 15 wherein the computer is further adapted to inform the user of a modification of the dietary regimen including a caloric restriction to bring a future measured body mass into conformance with model weight loss prediction.

17. The system of claim 15 wherein the caloric expenditure calculator is part of a wearable device.

18. The system of claim 17 wherein the wearable device is adapted to measure bio-impedance or heart rate.

19. The system of claim 15 wherein the weight scale, the caloric expenditure calculator, and the computer are connected to each other over a network.

20. The system of claim 15 wherein the computer is a cloud server that is remotely located with respect to both of the weight scale and the caloric expenditure calculator.
